(19) United States
(12) Patent Application Publication
(10) Pub. No.: US 2022/0370337 A1
(43) Pub. Date: Nov. 24, 2022

(54) COMPOSITION COMPRISING THE EXTRACT OF TREMELLA FUCIFORMIS CULTURE MEDIUM

(71) Applicant: DAEBONG LS, LTD, Incheon (KR)

(72) Inventors: Hyun Sook Yeom, Jeju-si (KR); Won Bo Oh, Seogwipo-si (KR); Ji Hye Kim, Seogwipo-si (KR); Hye Ja Lee, Seogwipo-si (KR); Jin Oh Park, Seoul (KR); Ji Won Lee, Seoul (KR); Chul Ho Kim, Daejeon (KR); Baek Rock Oh, Daejeon (KR); Min Soo Kim, Daejeon (KR); Sun Yeon Heo, Daejeon (KR); Jeong Woo Seo, Daejeon (KR)

(21) Appl. No.: 17/773,220

(22) PCT Filed: Sep. 29, 2020
(86) PCT No.: PCT/KR2020/013429

§ 371 Date: Apr. 29, 2022

(30) Foreign Application Priority Data
Nov. 1, 2019 (KR) 10-2019-0138381

(51) Int. Cl. A61K 8/9728; A61Q 19/00; A61Q 19/08

(52) U.S. Cl. CPC A61K 8/9728; A61Q 19/007; A61Q 19/08; A61K 2800/522

(57) ABSTRACT

The present invention relates to a composition including a Tremella fuciformis culture medium extract. The Tremella fuciformis culture medium extract according to the present invention not only has low cytotoxicity but also has the effects of increasing the production amount of a moisturizing factor and the production amounts of collagen and collagen fibers and inhibiting in vivo active oxygen species, and thus can be utilized as a natural product-derived moisturizing, anti-wrinkle, and anti-oxidant functional material, and can be applied in various ways in the beauty and food fields for skin condition improvement.

### TECHNICAL FIELD

The present invention relates to a composition including a Tremella fuciformis culture medium extract and, more particularly, to a cosmetic composition and a food composition capable of improving skin condition through skin moisturization, anti-wrinkle, or antioxidant.

This application is an application claiming priority of Korean patent application No. 10-2019-0138381 filed on Nov. 1, 2019, and all contents disclosed in the specification and drawings of the application are incorporated herein by reference.

### BACKGROUND ART

Tremella fuciformis belongs to Tremellaceae Family, Tremellales Order, Heterobasidiomycetes Class. It is a wood-rotting mushroom that occurs on old trees, dry branches, and stems of broad-leaved trees from spring to autumn and is distributed throughout the world including Korea, Japan, China, Europe, and America (Oh et al., 2006). Its common name is white jelly fungus or silver ear, and in Japan, it is called “rokikurage” (Ko, 2012). In addition, it has been known in China that from ancient times, eating wood ear mushrooms steadily activates the immune system, thereby preventing cancer and slowing aging, and preventing high blood pressure and arteriosclerosis (Chen and Cai, 2008). In a study on the physiological activity of Tremella fuciformis, it was found that the acidic polysaccharide isolated from the Tremella fuciformis exhibited antitumor activity of 37 to 64% against sarcoma 180 cells, a sarcoma cell line, in an animal model (Ukai et al., 1972). In addition, it has also been reported that Tremella fuciformis extract has the effect of improving body fat and suppressing the increase of cholesterol, and it is known that it has a long-term and short-term anti-stress effect (Cheng et al., 2002; Cheung, 1996; Ko et al., 2009).

Meanwhile, materials derived from various mushrooms such as black hoof mushroom, reishi mushroom, and oak mushroom, are being applied in many industrial fields. The related prior arts, the Korean patent No. 10-1924342 discloses the fine dust adsorption activity of the polysaccharides derived from fruiting body of Tremella fuciformis and the Korean patent application publication No. 10-2018-0124443 discloses the skin barrier strengthening effect of fruiting body extract of Tremella fuciformis. However, most of these mushroom-derived substances are fruiting body extracts, which are not only produced in small quantities, but also have a limitation in that the production cost is very high.

### PRIOR ART DOCUMENTS

### Patent Documents

(Patent Document 0001) Korean registered patent publication No. 10-1924342
(Patent Document 0002) Korean patent application publication No. 10-2018-0124443.

### DISCLOSURE

### Technical Problem

Therefore, in order to solve the problems of the prior arts as described above, the present inventors have completed the present invention by preparing a Tremella fuciformis culture medium extract and confirming its moisturization, anti-wrinkle, and antioxidant activity.

Accordingly, an objective of the present invention is to provide a composition including a Tremella fuciformis culture medium extract as an active ingredient.

### Technical Solution

In order to achieve the above objective, the cosmetic composition according to the present invention includes a Tremella fuciformis culture medium extract as an active ingredient.

Here, the Tremella fuciformis culture medium may be a mycelium culture medium.

In this case, the Tremella fuciformis culture medium may include a polysaccharide derived from the mycelium culture medium, and the polysaccharide may have a mannose content of to 60% by weight.

In addition, the Tremella fuciformis culture medium may be cultured for 6 to 48 hours.

In addition, the Tremella fuciformis culture medium may be cultured at 15 to 35° C.

In addition, the extract may be a polysaccharide derived from the Tremella fuciformis culture medium.

In addition, the extract may be extracted with water, C₁ to C₄ lower alcohol or a mixed solvent thereof.

In addition, the cosmetic composition may exhibit one or more efficacies selected from skin moisturization, anti-wrinkle, and antioxidant.

Meanwhile, the food composition according to the present invention includes a Tremella fuciformis culture medium extract as an active ingredient.

Here, the food composition may exhibit one or more efficacies selected from skin moisturization, anti-wrinkle, and antioxidant.

### Advantageous Effects

The Tremella fuciformis culture medium extract according to the present invention not only has low cytotoxicity but also has the effects of increasing the production amount of moisturizing factor and the production amounts of collagen and collagen fibers, and inhibiting intracellular reactive oxygen species. Thus, the extract can be used as a natural product-derived moisturizing, anti-wrinkle, and antioxidant functional material, and can be utilized in various ways in the beauty and food fields for skin condition improvement.

### DESCRIPTION OF DRAWINGS

FIG. 1 is a diagram illustrating the result of sugar analysis of a Tremella fuciformis mycelium culture medium extract according to the present invention;

FIG. 2 is a diagram illustrating the analysis result of the molecular weight of a polysaccharides derived from the Tremella fuciformis mycelium culture medium according to the present invention;

FIG. 3 is a diagram illustrating the result of confirming the production amount of the moisturizing factor hyaluronic acid (HA) of the Tremella fuciformis mycelium culture medium extract according to the present invention;

FIG. 4 is a diagram illustrating the result of confirming the production amount of the moisturizing factor aquaporin 3 (AQP3) of the Tremella fuciformis mycelium culture medium extract according to the present invention;

FIG. 5 is a diagram illustrating the result of quantifying the production amount of procollagen type 1 peptide according to the treatment of the Tremella fuciformis mycelium culture medium extract according to the present invention;

FIG. 6 is a diagram illustrating the result of observing collagen fiber generation through a fluorescence microscope according to the treatment of the Tremella fuciformis mycelium culture medium extract according to the present invention;

FIG. 7 is a diagram illustrating the result of measuring the active oxygen inhibitory activity according to the treatment of the Tremella fuciformis mycelium culture medium extract according to the present invention; and

FIG. 8 is a diagram illustrating the result of observing the intracellular reactive oxygen species according to the treatment of the Tremella fuciformis mycelium culture medium extract according to the present invention.

### BEST MODE

Hereinafter, preferred embodiments will be described in detail with reference to the drawings so that those of ordinary knowledge in the art to which the present invention pertains can easily practice the present invention. However, the present invention may be embodied in many different forms and should not be construed as being limited to the examples and drawings set forth herein.

The cosmetic composition for improving skin condition according to the present invention includes a Tremella fuciformis culture medium extract as an active ingredient.

The Tremella fuciformis culture medium extract according to the present invention not only has low cytotoxicity but also has the effects of increasing the production amount of moisturizing factor and the production amounts of collagen and collagen fibers, and inhibiting intracellular reactive oxygen species. Thus, it can be used as a natural product-derived skin moisturization, anti-wrinkle or antioxidant functional cosmetic material composition.

Tremella fuciformis is divided into mycelium and fruiting body. The mycelium, as a vegetative organ of a mushroom, corresponds to the root, stem, and leaf part of a general plant. It corresponds to the body of the mushroom that looks like a pear-colored fluff or thread. It spends most of its life as mycelium and lives a parasitic or saprophytic life. The fruiting body, as a reproductive organ of mushroom, corresponds to the flower of a general plant. After the mycelium accumulates sufficient nutrition, it grows at once to become a visible fruiting body when the climatic conditions are right. The mycelium and fruiting body of a mushroom differ in their constituent sugars and amino acids (Lee et al., 1999, “Characteristics of polysaccharide isolated from the fruiting body and cultured mycelia of Phellinus linteus IY001.” The Korean Journal of Mycology 27.6 (1999): 424-429).

Here, it is more preferable in term of effectiveness that the Tremella fuciformis culture medium is a Tremella fuciformis mycelium culture medium but is not limited thereto.

In this case, the Tremella fuciformis culture medium may include a polysaccharide derived from the mycelium culture medium. Preferably, the polysaccharide may have a mannose content of 20 to 60% by weight and more preferably, 30 to 50% by weight.

In addition, the Tremella fuciformis culture medium may be cultured for 6 to 48 hours and preferably for 12 to 36 hours but is not limited thereto.

When the culture time is less than 6 hours, the content and yield of the active ingredient may be low due to insufficient cultivation of the mycelium and when the culture time exceeds 48 hours, the culture efficiency of the mycelium may be reduced, or the mycelium may be deformed.

In addition, the Tremella fuciformis culture medium may be cultured at 15 to 35° C. and preferably at 20 to 30° C. but is not limited thereto.

When the culture temperature is less than 15° C., the content and yield of the active ingredient may be low due to insufficient cultivation of the mycelium and when the culture temperate exceeds 35° C., the culture efficiency of the mycelium may be reduced, or the mycelium may be deformed.

Meanwhile, the extract may be a polysaccharide derived from the Tremella fuciformis culture medium, which is a highly viscous polysaccharide that increases the procollagen synthetic ability and improves the moisturizing ability of skin with gelatin-like properties, thus is effective for improving skin wrinkle and has excellent anti-inflammatory effect, anti-aging effect, whitening effect, and collagen synthesis promoting effect. In addition, since it is stable from pH changes due to external harmful substances, it has an effect of calming skin irritation.

In addition, the extract may be extracted with water, C₁ to C₄ lower alcohol or a mixed solvent thereof so as to desirably act on improving skin condition. The lower alcohol may be methanol, ethanol, propanol, or butanol. The mixed solvent is not particularly limited, but preferably, be 20 to 80% by volume of an aqueous solution of methanol, ethanol, butanol, or propanol, and more preferably be 60 to 80% by volume of aqueous ethanol solution.

Meanwhile, the food composition for improving skin condition according to another aspect of the present invention includes a Tremella fuciformis culture medium extract as an active ingredient.

The Tremella fuciformis culture medium extract not only has low cytotoxicity but also has the effects of increasing the production amount of moisturizing factor and the production amounts of collagen and collagen fibers, and inhibiting intracellular reactive oxygen species. Thus, it can be used as a natural product-derived functional food composition for skin moisturization, anti-wrinkle or antioxidant.

Hereinafter, the present invention will be described in more detail with reference to the embodiments. The following examples are intended to illustrate the present invention, but the present invention is not limited by the following examples.

### Example 1. Preparation of Tremella fuciformis Mycelium Culture Medium Extract

A Tremella fuciformis mycelium culture medium extract was prepared by extracting the culture medium of culturing Tremella fuciformis mycelium with ethanol. Specifically, the mycelium was separated from the Tremella fuciformis, and the separated mycelium was cultured under the condition of 300 ml of MCM (Mushroom Complete Medium) medium, 25° C. and 180 rpm for 24 hours. The cultured mycelium was centrifuged to obtain a supernatant. The separated supernatant and ethanol were mixed (separated supernatant:ethanol=1:3 (volume ratio)), and then cultured overnight with stirring at 4° C. The cultured mixture was centrifuged (at 12,000 rpm) to obtain a precipitate, that is, a polysaccharide derived from the Tremella fuciformis mycelium culture medium. The obtained polysaccharide derived from the Tremella fuciformis mycelium culture medium was diluted with purified water (polysaccharide:purified water=1:99) and filtered to prepare a Tremella fuciformis mycelium culture medium extract.

### Experimental Example 1. Sugar Analysis of Extract Derived from Tremella fuciformis Mycelium Culture Medium

1-1. Analysis of Polysaccharide Content of Extract Derived from Tremella Fuciformis Mycelium Culture Medium

1-1-1. Hydrolysis of Neutral Sugar

100 μg of a polysaccharide derived from the Tremella fuciformis mycelium culture medium and 400 μl of 2 M TFA (Trifluoroacetic acid) were each added to a microcentrifuge tube and hydrolyzed at 100° C. for 4 hours. After cooling at room temperature, it was dried using a vacuum centrifugal concentrator, and the polysaccharide derived from the Tremella fuciformis mycelium culture medium was dissolved in 2 ml of tertiary distilled water and filtered through 02. Um filter.

1-1-2. Hydrolysis of Glucuronic Acid

100 μg of a polysaccharide derived from the Tremella fuciformis mycelium culture medium and 400 μl of 2 M TFA (Trifluoroacetic acid) were each added to a microcentrifuge tube and hydrolyzed at 100° C. for 6 hours. After cooling at room temperature, it was dried using a vacuum centrifugal concentrator, and the sample was dissolved in 1 ml of tertiary distilled water and filtered through 02. Um filter.

1-1-3. Sugar Analysis Using HPAEC (High-Performance Anion-Exchange Chromatography)

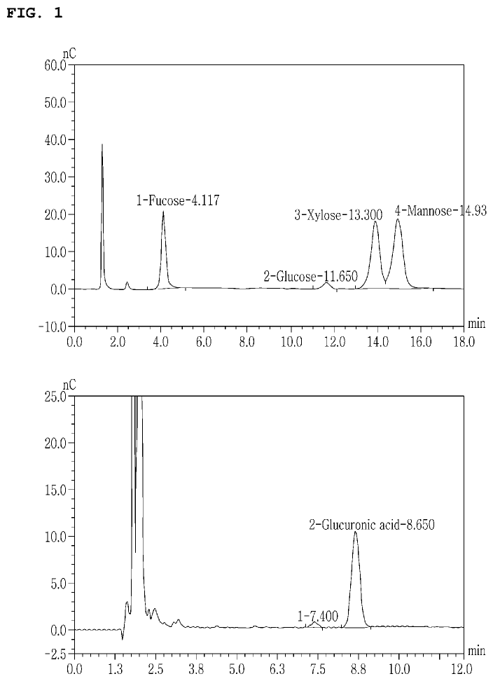

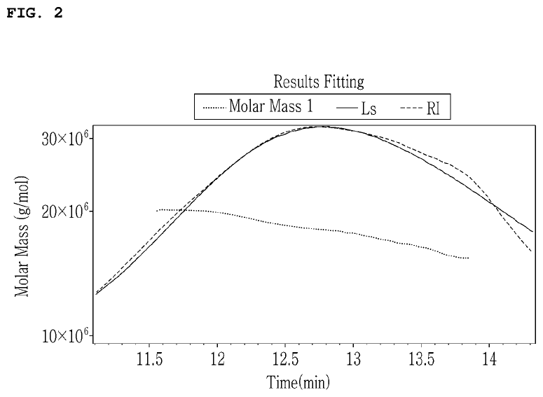

The sugars hydrolyzed in Experimental Examples 1-1-1 and 1-1-2 were analyzed using HPAEC. Specific HPAEC analysis conditions are shown in Table 1 below and the sugar analysis results are shown in FIGS. 1 and 2.

TABLE 1

| | Neutral sugar | Glucuronic acid |
|---|---|---|
| Mode | Anion-exchange chromatography | |
| Detection | Integrated amperometry | |
| Solvent | 2 mM NaOH | 100 mM NaOH/150 |
| | | mM sodium acetate |
| Velocity of | 0.5 | 1.0 |
| flow (ml/min) | | |
| Column | CarboPac PA20 column | CarboPac PA1 column |
| | (3 × 150 mm, Dionex, | (4 × 250 mm, Dionex, |
| | 060142) and | 035391) and guard |
| | AminTrap (3 ×30 mm, | column(4 × 50 mm, |
| | Dionex 060146) | Dionex, 043096) |
| Temperature, | Column oven, 30; | |
| ° C. | Autosampler, 8 | |

TABLE 2

| | Tremella fuciformis | Tremella fuciformis |
|---|---|---|
| | mycelium culture | fruiting body culture |
| | medium extract | medium extract |
| Sugar | Content (% by weight) | Content (% by weight) |
| Fucose | 17.1 | 11.2 |
| Glucose | 1.3 | 50.5 |
| Xylose | 21.0 | 15.2 |
| Annose | 40.3 | 8.5 |
| Glucuronic acid | 17.1 | 13.0 |
| Total | 96.8 | 98.4 |

As shown in Table 2, it was confirmed that the glucose content of the polysaccharide derived from the Tremella fuciformis mycelium culture medium was lower than that of the polysaccharide derived from the Tremella fuciformis fruiting body culture medium. On the other hand, it was confirmed that the mannose content of the polysaccharide derived from the Tremella fuciformis mycelium culture medium was about 8 times higher than that of the polysaccharide derived from the Tremella fuciformis fruiting body culture medium. Mannose, to which α-mannan belongs, is a polysaccharide that induces skin and intestinal immunity by binding to Dectin-2 receptor present in the cell membrane.

1-2. Analysis of Polysaccharide Molecular Weight of Extract Derived from Tremella fuciformis Mycelium Culture Medium

The average molecular weight of polysaccharides derived from the Tremella fuciformis mycelium culture medium was analyzed using SEC column (TSK-gel-GMPWXL, 30 cm×7.8 mm, 13 μm, Tosoh) in a high-performance liquid chromatography-MALS (Multi-angle light scattering) system (Wyatt Technology, Santa Barbara, Calif.). Polysaccharides derived from the Tremella fuciformis mycelium culture medium were dissolved in phosphate buffer (pH 7.3 to 7.5) to a concentration of 3 mg/ml. Analysis samples were analyzed using high performance liquid chromatography flow rate of 0.5 ml/min, injection volume of 100 μl, mobile phase phosphate buffer saline (pH 7.3 to pH 7.5), detector DAWN HELEOS II, and Optilab rEX. For molecular weight calculation, the dn/dc value (specific refractive index increment) was determined to be 0.15 ml/g with reference to the literature. ASTRA 6 software (Wyatt Technology) was used to calculate the average molecular weight of polysaccharides derived from the Tremella fuciformis mycelium culture medium. FIG. 2 illustrates the results of high-performance liquid chromatography-MALS using polysaccharides derived from the Tremella fuciformis mycelium culture medium.

As shown in FIG. 2, it was confirmed that the average molecular weight of the polysaccharide derived from the Tremella fuciformis mycelium culture medium was 1.79×10⁶ (±0.721%).

### Experimental Example 2. Skin Cell Culture

2.1. Skin Keratinocyte Culture

HaCat cells, which are skin keratinocyte, were purchased from Cell Line Service GmbH (Germany) and used. Using DMEM (Dulbecco's Modified Eagle's Medium) culture containing 1% penicillin-streptomycin and 10% fetal bovine serum (FBS), they were cultured in 37° C., 5% CO₂ thermostat, and subculturing was performed at intervals of 2 to 3 days.

2-2. Skin Fibroblast Culture

Normal Human Dermal Fibroblasts (NHDF) cells, which are dermal fibroblasts, were purchased from Lonza (Lonza Walkersville, Inc) and were cultured in 37° C., 5% CO₂ thermostat using FBM (Fibroblast Basal Medium) medium containing 0.1% hFGF-B, insulin, GA-1000, and 2% fetal bovine serum, and subculturing was performed at intervals of 3 to 5 days.

### Experimental Example 3. Cytotoxicity Evaluation of Tremella fuciformis Mycelium Culture Medium Extract

EZ-cytox assay is a typical cytotoxicity evaluation method that measures cell viability using the principle that water solution tetrazolium salt (WST) reacts with dehydrogenase of living cells to generate orange water-soluble formazan.

3-1. Cytotoxicity Evaluation of Skin Keratinocyte

To confirm toxicity in cells, HaCaT cells were aliquoted in a 96-well plate at a concentration of 5×10⁴ cells/ml and cultured under condition of 37° C., 5% CO₂ for 18 hours. After exchanging the cultured cells with a serum-free medium, the Tremella fuciformis mycelium culture medium extracts prepared in Example 1 were treated with various concentrations (0.1, 0.5, 1.0, 2.0, and 4.0 v/v %). Then, EZ-cytox was added to each well and reacted under condition of 37° C., 5% CO₂ for 30 minutes. Absorbance was measured at 450 nm of each well using a microplate reader. The average absorbance value for each sample group was obtained, and the cell viability was evaluated by comparing this value with the absorbance value of the control group. The results of the cytotoxicity evaluation of the Tremella fuciformis mycelium culture medium extracts against skin keratinocyte were shown in Table 3 below.

TABLE 3

| | Cytotoxicity | |
|---|---|---|
| | Treatment | HaCaT cell |
| Classification | concentration (%) | growth rate (%) |
| Untreated group | — | 100 |
| Extracts of tremella | 0.1 | 99.8 ± 4.3 |
| fuciformis mycelium | 0.5 | 98.6 ± 2.1 |
| culture medium | 1.0 | 95.7 ± 6.5 |
| (Example 1) | 2.0 | 94.8 ± 2.4 |
| | 4.0 | 87.0 ± 4.7 |

As shown in Table 3 above, it was confirmed that the Tremella fuciformis mycelium culture medium extracts has low cytotoxicity to skin keratinocytes.

3-2. Cytotoxicity Evaluation of Skin Fibroblast

Normal Human Dermal Fibroblasts (NHDF), which are skin fibroblasts, were aliquoted in a 96-well plate at a concentration of 1×10⁴ cells/ml. The cytotoxicity of the Tremella fuciformis mycelium culture medium extracts was evaluated through EZ-cytox assay of Experimental Example 2-1. The results of the cytotoxicity evaluation of the Tremella fuciformis mycelium culture medium extracts against skin fibroblast were shown in Table 4 below.

TABLE 4

| | Cytotoxicity | |
|---|---|---|
| | Treatment | NHDF cell |
| Classification | concentration (%) | growth rate (%) |
| Untreated group | — | 100 |
| Extracts of Tremella | 0.1 | 104.4 ± 3.8 |
| fuciformis mycelium | 0.5 | 97.4 ± 5.2 |
| culture medium | 1.0 | 97.5 ± 4.2 |
| (Example 1) | 2.0 | 91.1 ± 4.6 |
| | 4.0 | 78.9 ± 5.0 |

As shown in Table 4 above, it was confirmed that the Tremella fuciformis mycelium culture medium extracts has low cytotoxicity to skin fibroblast.

### Experimental Example 4. Confirmation of Moisturizing Effect of Tremella fuciformis Mycelium Culture Medium Extract

4-1. Confirmation of Effect of Increasing Production Amount of Hyaluronic Acid (HA), which is a Moisturizing Factor of Tremella fuciformis Mycelium Culture Medium Extract

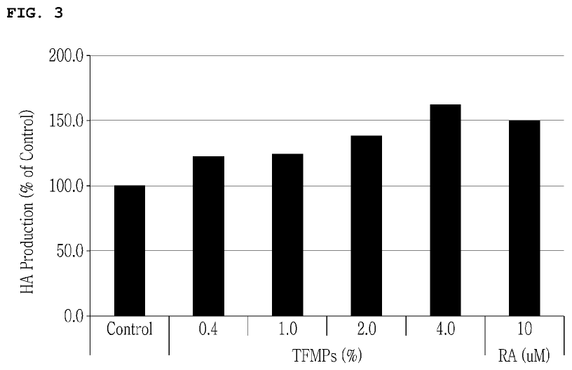

HaCaT cells were aliquoted in a 24-well plate at 1.0×10⁵ cells/ml and cultured under condition of 37° C., 5% CO₂ for 18 hours. After exchanging the medium of the cultured cells with a serum-free DMEM medium, the Tremella fuciformis mycelium culture medium extracts prepared in Example 1 were each treated with the concentrations, 0.1, 0.5, and 1.0 v/v % and cultured for 24 hours. Then, the production amount of hyaluronic acid of the culture medium extracts of each experimental group was measured. The control group was treated with retinoic acid (RA) at a concentration of 10 μm. The production amount of hyaluronic acid was measured using HA-ELISA kit (Cusabio Biotechnology Co., Ltd), and the experiment was performed by the method provided by the manufacturer. FIG. 3 illustrates the results of measuring the production amount of hyaluronic acid according to the treatment of the Tremella fuciformis mycelium culture medium extract.

As shown in FIG. 3, it was confirmed that the Tremella fuciformis mycelium culture medium extract increases the production of hyaluronic acid, which is a moisturizing factor, in a concentration-dependent manner. In addition, it can be known that the Tremella fuciformis mycelium culture medium produces hyaluronic acid at a level similar to that of the retinoic acid control group.

4-2. Confirmation of Effect of Increasing Production Amount of Aquaporin 3 (AQP3), which is a Moisturizing Factor of Tremella fuciformis Mycelium Culture Medium Extract

HaCaT cells were aliquoted in a 24-well plate at 1.0×10⁵ cells/ml and cultured under condition of 37° C., 5% CO₂ for 18 hours. After exchanging the medium of the cultured cells with a serum-free DMEM medium, the Tremella fuciformis mycelium culture medium extracts prepared in Example 1 were each treated with the concentrations, 0.1, 0.5, and 1.0 v/v % and cultured for 24 hours. Then, after dissolving the cells of each experimental group, a protein was taken, and the production amount of aquaporin 3 in the culture medium extract was measured. The control group was treated with retinoic acid (RA) at a concentration of 10 μm. The production amount of aquaporin 3 was measured using AQP3-ELISA kit (Cusabio Biotechnology Co., Ltd), and the experiment was performed by the method provided by the manufacturer. FIG. 4 illustrates the results of measuring the production amount of aquaporin 3 according to the treatment of the Tremella fuciformis mycelium culture medium extract.

As shown in FIG. 4, it was confirmed that the Tremella fuciformis mycelium culture medium extract increases the production of aquaporin 3, which is a moisturizing factor, in a concentration-dependent manner.

As a result of the experiment, it was confirmed that the Tremella fuciformis mycelium culture medium extract prepared in Example 1 increases the production of hyaluronic acid and aquaporin 3. The result is believed to be since the Tremella fuciformis mycelium culture medium extract contains mannose at a high concentration. Therefore, it can be confirmed that the Tremella fuciformis mycelium culture medium extract has a high potential to be used as a nature derived moisturizing factor.

### Experimental Example 5. Confirmation of Anti-Wrinkle Activity of Tremella fuciformis Mycelium Culture Medium Extract

5-1. Confirmation of Effect of Procollagen Type 1 Peptide (PIP) Production of Tremella fuciformis Mycelium Culture Medium Extract

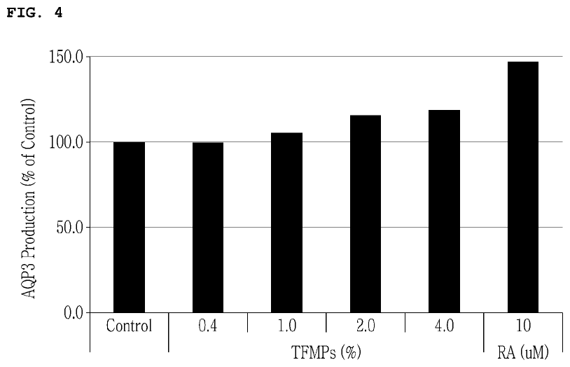

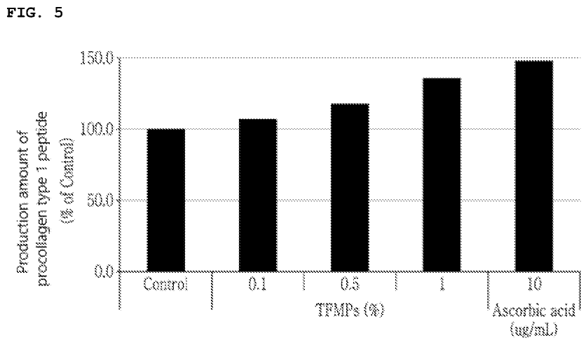

NHDF cells, which are skin fibroblasts, were aliquoted in a 24-well plate at 2×10⁴ cells/ml and then cultured under cell culture condition for 24 hours. After culturing, it was exchanged with the serum-free FBM medium, the Tremella fuciformis mycelium culture medium extract prepared in Example 1 was treated at concentrations of 0.1, 0.5, 1.0 v/v %, respectively, and cultured for 24 hours. After culturing, the supernatant of each group was taken, and the amount of procollagen released in the medium was measured. The control group was treated with ascorbic acid at a concentration of 10 μg/ml instead of the extract. The amount of procollagen was measured using a procollagen type I peptide (PIP) EIA kit (Takara Biomedical Co.), and the amount of collagen was quantified by measuring absorbance at a wavelength of 450 nm according to the manufacturer's manual. FIG. 5 illustrates the results of quantifying the amount of procollagen type 1 peptide according to the treatment of the Tremella fuciformis mycelium culture medium extract.

As shown in FIG. 5, it was confirmed that the Tremella fuciformis mycelium culture medium extract increases collagen synthesis in a concentration-dependent manner.

5-2. Confirmation of Effect of Collagen Fiber Production of Tremella fuciformis Mycelium Culture Medium Extract

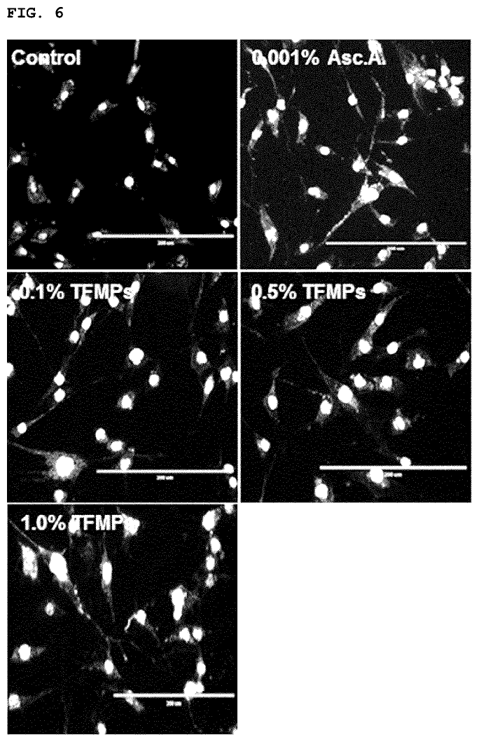

NHDF cells, which are skin fibroblasts, were aliquoted in a 24-well plate at 2×10⁴ cells/ml and then cultured under cell culture condition for 24 hours. After culturing, cells were washed with HEPES-BSS (HEPES Buffered Saline Solution), and intracellular collagen fibers were stained according to the immunofluorescent staining method. The degree of fluorescence expression of the stained cells was measured using a fluorescence microscope. FIG. 6 illustrates the result of confirming the collagen fiber production according to the treatment of the Tremella fuciformis mycelium culture medium extract.

As shown in FIG. 6, it was confirmed that the Tremella fuciformis mycelium culture medium extract increases the production of collagen fiber in skin fibroblasts in a concentration-dependent manner.

As a result of the experiment, the Tremella fuciformis mycelium culture medium extract prepared in Example 1 increases the production of collagen synthesis and collagen fiber, therefore it was confirmed that the Tremella fuciformis mycelium culture medium extract has a high potential to be used as an anti-wrinkle material.

### Experimental Example 6. Confirmation of Antioxidant Effect of Tremella fuciformis Mycelium Culture Medium Extract

6-1. Measurement of Effect of Inhibiting Intracellular Reactive Species (ROS)

In order to measure changes in intracellular reactive oxygen species, HaCaT cells were inoculated into a 24-well plate with 1.0×10⁵ cells/well and cultured for 24 hours, and then the Tremella fuciformis mycelium culture medium extract prepared in Example 1 was pretreated for 24 hours. After washing the pretreated cells with PBS (phosphate buffered saline), the experiment was performed according to the manufacturer's manual using Intracellular ROS assay kit (Green Fluorescence). FIG. 7 illustrates the results of measuring the effect of inhibiting intracellular reactive oxygen species.

As shown in FIG. 7, it was confirmed that the Tremella fuciformis mycelium culture medium extract has high free radical scavenging activity in a concentration-dependent manner. In particular, in the experimental group treated with a concentration of 2.0 v/v % of the Tremella fuciformis mycelium culture medium extract, it can be confirmed that about more than 40% of free radicals were removed.

6-2. Measurement of Effect of Inhibiting Intracellular Reactive Species (ROS) Through Imaging

To visualize changes in intracellular reactive oxygen species (ROS), after HaCaT cells, which are skin keratinocytes, were inoculated in a 24-well plate at a concentration of 1.0×10⁵ cells/well and cultured for 24 hours, the Tremella fuciformis mycelium culture medium extracts prepared in Example 1 were treated with various concentrations (0.1, 0.5, 1.0, and 2.0 v/v %) and then cultured for 24 hours. After washing the cultured cells with phosphate buffered saline (PBS), H₂O₂ was treated at a concentration of 300 μm and further cultured for 3 hours. DCF-DA (dichlorofluorescein diacetate), a dye for measuring intracellular reactive oxygen, was added at a concentration of 50 μm and cultured for 1 hour. After washing the cultured cells with PBS, the degree of fluorescence expression of the washed cells was measured using a fluorescence microscope. FIG. 8 illustrates the results of measuring the intracellular reactive oxygen species of the Tremella fuciformis mycelium culture medium extract.

As shown in FIG. 8, it was confirmed that the Tremella fuciformis mycelium culture medium extract inhibits the production of intracellular reactive oxygen species in a concentration-dependent manner. In particular, the experimental group treated the Tremella fuciformis mycelium culture medium extract at concentrations of 0.5, 1.0, and 2.0 v/v % has excellent effect of inhibiting intracellular reactive oxygen species.

As a result of the experiment, the Tremella fuciformis mycelium culture medium extract prepared in Example 1 has a remarkable effect of inhibiting intracellular oxygen species, therefore it was confirmed that the Tremella fuciformis mycelium culture medium extract has a high potential to be used as an antioxidant material.

Comprehensively, the present inventors prepared a Tremella fuciformis mycelium culture medium extract and confirmed that the extract not only has low cytotoxicity but also has the effects of increasing the production amount of moisturizing factor and the production amounts of collagen and collagen fibers and inhibiting intracellular reactive oxygen species. This means that the Tremella fuciformis mycelium culture medium extract of the present invention can be used as a natural product-derived moisturizing, anti-wrinkle, and antioxidant functional material and thus, the Tremella fuciformis mycelium culture medium extract of the present invention can be utilized in various ways in the beauty and food fields for skin condition improvement.

Hereinafter, the present invention will be described in more detail with reference to the preparation examples. The following examples are only for illustrating the present invention and the scope of the present invention is not construed as being limited by the preparation examples.

### Preparation Example 1. Preparation of Cosmetic Composition for Improving Skin Condition

1-1. Preparation of Softening Lotion (Skin Lotion)

Tremella fuciformis mycelium culture medium extract 0.5% by weight

beta-1,3-glucan 1.0% by weight

butylene glycol 2.0% by weight

propylene glycol 2.0% by weight

carboxy vinyl polymer 0.1% by weight

PEG-12 nonylphenyl ether 0.2% by weight

polysolveate 80 0.4% by weight

ethanol 10.0% by weight

triethanolamine 0.1% by weight

Appropriate amount of preservatives, colors, and fragrances

purified water to 100% by weight

1-2. Preparation of Nourishing Lotion (Milk Lotion)

Tremella fuciformis mycelium culture medium extract 0.5% by weight

beta-1,3-glucan 1.0% by weight

beeswax 4.0% by weight

polysolveate 60 1.5% by weight

sorbitan sesquioleate 1.5% by weight

liquid paraffin 0.5% by weight

caprylic/caprylic triglyceride 5.0% by weight

glycerin 3.0% by weight

butylene glycol 3.0% by weight

propylene glycol 3.0% by weight

carboxy vinyl polymer 0.1% by weight

triethanolamine 0.2% by weight

Appropriate amount of preservatives, colors, and fragrances

purified water to 100% by weight

1-3. Preparation of Nourishing Cream

Tremella fuciformis mycelium culture medium extract 1.0% by weight

beta-1,3-glucan 5.0% by weight

beeswax 10.0% by weight

polysolveate 60 1.5% by weight

PEG-60 hydrogenated castor oil 2.0% by weight

sorbitan sesquioleate 0.5% by weight

liquid paraffin 10.0% by weight

squalene 5.0% by weight

caprylic/caprylic triglyceride 5.0% by weight

glycerin 5.0% by weight

butylene glycol 3.0% by weight

propylene glycol 3.0% by weight

triethanolamine 0.2% by weight

Appropriate amount of preservatives, colors, and fragrances

purified water to 100% by weight

### Preparation Example 2. Preparation of Food Composition for Improving Skin Condition

2-1. Preparation of Health Food

Tremella fuciformis mycelium culture medium extract 100 mg

appropriate amount of vitamin mixture

vitamin A acetate 70 g

vitamin E 1.0 mg

vitamin B1 0.13 mg

vitamin B2 0.15 mg

vitamin B6 0.5 mg

vitamin B12 0.2 g

vitamin C 10 mg

biotin 10 g

nicotinamide 1.7 mg

folic acid 50 g

calcium pantothenate 0.5 mg

appropriate amount of mineral mixture

ferrous sulfate 1.75 mg

zinc oxide 0.82 mg

magnesium carbonate 25.3 mg

monopotassium phosphate 15 mg

dipotassium phosphate 55 mg

potassium citrate 90 mg

potassium carbonate 100 mg

magnesium chloride 24.8 mg

Although the composition ratio of the vitamin and mineral mixture was prepared by mixing components suitable for relatively healthy food in a preferred embodiment, the mixing ratio may be arbitrarily modified, and after mixing the components according to a conventional health food preparing method, granules can be prepared, and can be used for preparing health food composition according to a conventional method.

2-2. Preparation of Health Drink

Tremella fuciformis mycelium culture medium extract 100 mg

vitamin C 15 g

vitamin E (powder) 100 g

ferrous lactate 19.75 g

zinc oxide 3.5 g

nicotinamide 3.5 g

vitamin A 0.2 g

vitamin B1 0.25 g

vitamin B2 0.3 g

appropriate amount of water

After mixing the ingredients according to a conventional method of preparing a health drink, the mixture was stirred and heated at 85° C. for about 1 hour. Then, the resulting solution was filtered and obtained in a sterilized 2 L container, sealed and sterilized, then refrigerated. It was used for preparing the health drink according to the present invention.

Although the composition ratio was prepared by mixing components suitable for relatively favorite beverages in a preferred embodiment, the mixing ratio may be arbitrarily modified according to regional and national preferences such as demanding class, demanding country, use, etc.

The description is merely illustrative of the present invention, and those of ordinary skill in the art to which the present invention pertains will understand that the present invention may be implemented in a modified form without departing from the essential features of the present invention. Therefore, the disclosed embodiments and experimental examples should be considered in an illustrative rather than a restrictive point of view. The scope of this invention is set forth in the claims, not in the aforementioned description, and any differences within the scope equivalent thereto should be construed as being included in the present invention.

1. A cosmetic composition comprising a Tremella fuciformis culture medium extract as an active ingredient.

2. The composition of claim 1, wherein the Tremella fuciformis culture medium is a mycelium culture medium.

3. The composition of claim 1, wherein the Tremella fuciformis culture medium comprises a polysaccharide derived from the mycelium culture medium, and
the polysaccharide has a mannose content of 20 to 60% by weight.

4. The composition of claim 1, wherein the Tremella fuciformis culture medium is cultured for 6 to 48 hours.

5. The composition of claim 1, wherein the Tremella fuciformis culture medium is cultured at 15 to 35° C.

6. The composition of claim 1, wherein the extract is a polysaccharide derived from the Tremella fuciformis culture medium.

7. The composition of claim 1, wherein the extract is extracted with water, C₁-C₄ lower alcohol, or a mixed solvent thereof.

8. The composition of claim 1, wherein the cosmetic composition exhibits one or more effects selected from skin moisturization, anti-wrinkle, and antioxidant.

9. A food composition comprising a Tremella fuciformis culture medium extract as an active ingredient.

10. The composition of claim 9, the food composition exhibits one or more effects selected from skin moisturization, anti-wrinkle, and antioxidant.
